(19) United States
(12) Patent Application Publication
(10) Pub. No.: US 2004/0224989 A1
(43) Pub. Date: Nov. 11, 2004

(54) S-lansoprazole compositions and methods

(76) Inventors: Timothy J. Barberich, Concord, MA (US); William E. Yelle, Billerica, MA (US); Paul D. Rubin, Sudbury, MA (US)

Correspondence Address: HESLIN ROTHENBERG FARLEY & MESITI PC, 5 COLUMBIA CIRCLE, ALBANY, NY, 12203, US

(21) Appl. No.: 10/863,532

(22) Filed: Jun. 8, 2004

Related U.S. Application Data
(63) Continuation of application No. 09/854,065, filed on May 11, 2001, now abandoned.
Application No. 09/854,065 is a continuation of application No. 09/240,262, filed on Jan. 29, 1999, now abandoned.

(51) Int. Cl. A61K 31/4439

(52) U.S. Cl. 514/338

(57) ABSTRACT

Methods and compositions are disclosed utilizing optically pure (−) lansoprazole for the treatment of ulcers in humans while substantially reducing the concomitant liability of adverse effects associated with the racemic mixture of lansoprazole. The optically pure (−) isomer is also-useful for the treatment of gastroesophageal reflux. (−) Lansoprazole is an inhibitor of H⁺ release and is therefore useful in the treatment of other conditions related to gastric hypersecretion such as Zollinger-Ellison Syndrome.

### CROSS REFERENCE TO RELATED APPLICATIONS

This application claims the priority of U.S. provisional application 60/073,141, filed Jan. 30, 1998, and 60/107,460, filed Nov. 5, 1998, the disclosures of which are incorporated herein by reference.

### FIELD OF THE INVENTION

This invention relates to compositions of matter containing lansoprazole. The invention also relates to methods of treating and preventing ulcers, treating other conditions related to gastric hypersecretion, and treating psoriasis.

### BACKGROUND OF THE INVENTION

Racemic lansoprazole is an orally active, potent, irreversible inhibitor of H⁺, K⁺-ATPase. The compound is one of the class of compounds known as gastric “proton pump” inhibitors. These compounds are weak organic bases which diffuse passively from the plasma into the acid-containing intracellular canaliculi of gastric parietal cells. At the low pH found in the lumen of these canaliculi, the protonated compounds rearrange to form pyridinium sulfenamides, which react with sulfhydryl groups present on the ATPase localized in the membranes lining the intracellular canaliculi. The alkylation of the sulfhydryl inhibits the ability of the enzyme to catalyze the secretion of H⁺ into the lumen in exchange for K⁺ ions. This inhibition results in an overall reduction in hydrochloric acid secretion by the parietal cells into the cavity of the stomach, thus increasing intragastric pH. As a consequence of reduced acidity in the stomach, the activity of the proteolytic enzyme pepsin is also markedly decreased. Because the proton pump is the final step in acid production and the compounds of this class combine covalently with the associated H⁺, K⁺-ATPase, a profound and prolonged inhibition of gastric acid secretion can be achieved.

Proton pump inhibitors have also been reported as useful in treating psoriasis. [See PCT application WO95/18612]

The Cₘₐₓ of racemic lansoprazole is at about 1.7 hours in humans and the serum half-life is about 1.5 hours, but this does not reflect the duration of the acid inhibitory effect, which is about 24 hours. Racemic lansoprazole is comparable to omeprazole in its effects on hepatic drug metabolizing enzyme systems.

Although no cardiovascular or obvious physical sequelae of elevated gastrin have been observed in humans on administration of racemic lansoprazole, fasting serum gastrin levels are significantly elevated. This is cause for concern because prolonged elevated serum gastrin appears to be associated with diffuse and focal enterochromaffin-like cell hyperplasia and focal neoplasia (carcinoids) in rats. [Larsson et al. Gastroenterolocy 90, 391-399 (1986)]. Thus, despite its advantages, adverse effects of racemic lansoprazole may remain, including, but not limited to, some incidence of hepatocellular neoplasia and gastric carcinoids on long-term therapy, and headache, diarrhea and skin alterations on acute therapy. There has also been some concern about the inhibition of cytochrome P450 enzymes by racemic lansoprazole [Kromer Digestion 56, 443-454 (1995)]; this effect would lead to adverse drug-drug interactions.

The following adverse events have been reported in lansoprazole-treated patients: Body as a Whole —asthenia, candidiasis, chest pain (not otherwise specified), edema, fever, flu syndrome, halitosis, infection (not otherwise specified), malaise; Cardiovascular System—angina, cereborvascular accident, hypertension/hypotension, myocardial infarction, palpitations, shock (circulatory failure), vasodilation; Digestive System—melena, anorexia, bezoar, cardiospasm, cholelithiasis, constipation, dry mouth/thirst, dyspepsia, dysphagia, eructation, esophageal stenosis, esophageal ulcer, esophagitis, fecal discoloration, flatulence, gastric nodules/fundic gland polyps, gastroenteritis, gastrointestinal hemorrhage, hematemesis, increased appetite, increased salivation, rectal hemorrhage, stomatitis, tenesmus, ulcerative colitis, vomiting; Endocrine System—diabetes mellitus, goiter, hyperglycemia/hypoglycemia, Hematologic and Lymphatis System—anemia, hemolysis; Metabolic and Nutritional Disorders—gout, weight gain/loss; Musculoskeletal System—arthritis/arthralgia, musculoskeletal pain, myalgia; Nervous System—agitation, amnesia, anxiety, apathy, confusion, depression, dizziness/syncope, hallucinations, hemiplegia, aggravated hostility, decreased libido, nervousness, paresthesia, thinking abnormality; Respiratory System—asthma, bronchitis, cough increased, dyspnea, epistaxis, hemoptysis, hiccup, pneumonia, upper respiratory inflammation/infection; Skin and Appendages—acne, alopecia, pruritis, rash, urticaria, Special Senses—amblyopia, deafness, eye pain, visual field defect, otitis media, taste perversion, tinnitus; Urogenital System—abnormal menses, albuminuria, breast enlargement/gynecomastia, breast tenderness, glycosuria, hematuria, impotence, kidney calculus.

It would therefore be particularly desirable to find a compound with the advantages of the racemic mixture of lansoprazole which would not have the aforementioned disadvantages.

### SUMMARY OF THE INVENTION

This invention relates to the use of optically pure S(−) lansoprazole for treating ulcers of the stomach, duodenum and esophagus, gastroesophageal reflux diseases, Zollinger-Ellison Syndrome, and other disorders including those that would benefit from an inhibitory action on gastric acid secretion. S(−) Lansoprazole inhibits the H⁺, K⁺-ATPase associated with the gastric proton pump and the resulting secretion of gastric acid by parietal cells providing therapy in diseases associated with gastric hyperacidity. The invention also relates to a method of treating psoriasis using optically pure S(−) lansoprazole. Optically pure (−) lansoprazole provides this treatment while substantially reducing adverse effects, including, but not limited to, hepatocellular neoplasia, gastrin hypersecretion, gastric neoplasms or carcinoids, headache, diarrhea and skin alterations which are associated with the administration of the racemic mixture of lansoprazole.

The invention also relates to certain oral pharmaceutical compositions containing the S(−) isomer of lansoprazole.

### DETAILED DESCRIPTION OF THE INVENTION

The active compound of these compositions and methods is an optical isomer of lansoprazole. The preparation of racemic lansoprazole is described in U.S. Pat. Nos. 4,628,098 and 4,689,333. The medicinal chemistry and clinical aspects of racemic lansoprazole have been reviewed by Garnett [Ann. Pharmacother. 30, 1425-1436 (1996)], by Langtry and Wilde [Drugs 54, 473-500 1997)] and by Barradell et al. [Drugs 44, 225-250(1992)]. Chemically, the active compound is the (−) isomer of 2-[3-methyl-4-(2,2,2-trifluoroethoxy)pyrid-2-yl]methylsulfinyl-benzimidazole(I), hereinafter referred to as lansoprazole.

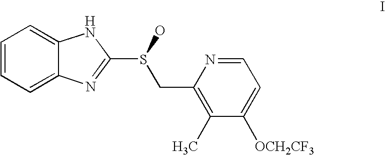

(−) Lansoprazole, which is the subject of the present invention, is not presently commercially available; only the 1:1 racemic mixture is commercially available as Prevacid®.

Syntheses of R (+) lansoprazole and S(−) lansoprazole by asymmetric oxidation and by bioreduction are described in PCT applications WO 9602535 and 9617077, respectively, the disclosures of which are incorporated herein by reference. The enrichment of single enantiomers by crystallization of the racemate from non-racemic mixtures is described in PCT application WO 97/02261, the disclosure of which is also incorporated herein by reference.

The pharmacology of the individual enantiomers in canine parietal cells and gastric microsomes has been reported by Nagaya et al. [Biochem. Pharmacol. 42, 1875-1878 (1991)], who concluded that “the effects of the (+) and (−) enantiomer of lansoprazole on acid formation stimulated by db-cAMP in isolated parietal cells were almost identical.” Similarly, inhibition of ATPase activity in gastric microsomes by the two enantiomers did not differ significantly over the range of concentrations tested.

It has now been discovered that the optically pure (−) isomer of lansoprazole is a superior agent for treating ulcers of the stomach, duodenum and esophagus, gastroesophageal reflux diseases, Zollinger-Ellison. Syndrome, psoriasis and other disorders, including those that would benefit from an inhibitory action on H⁺, K⁺-ATPase in that it provides this effective treatment while substantially reducing the adverse effects of racemic lansoprazole including, but not limited to, hepatocellular neoplasia, gastric carcinoids, headache, diarrhea and skin alterations. The S(−) isomer of lansoprazole is also a superior agent for treating ulcers and other disorders by virtue of its lessened liability for drug-drug interactions and its greater predictability of dosage among patients, as discussed below. Surprisingly, it also shows a longer duration, a higher AUC (area under the curve—a composite measure of efficacy and duration), and a more rapid onset as a result of lower first pass metabolism.

The present invention encompasses a method of treating ulcers, which comprises administering to a human in need of such therapy, an amount of (−) lansoprazole, or a pharmaceutically acceptable salt thereof, substantially free of its (+) stereoisomer, said amount being sufficient to alleviate the symptoms of ulcers. The method substantially reduces the concomitant liability of adverse effects associated with the administration of the racemic compound by providing an amount which is insufficient to cause the adverse effects associated with the racemic mixture of lansoprazole.

The present invention also encompasses an oral antiulcer composition for the treatment of a human in need of antiulcer therapy, which comprises a pharmaceutically acceptable carrier for oral administration and a therapeutically effective amount of (−) lansoprazole, or a pharmaceutically acceptable salt thereof, substantially free of its (+) stereoisomer. Preferably the composition is in the form of a tablet or capsule and the amount of (−) lansoprazole in the tablet or capsule is 15, 30 or 60 mg.

The present invention further encompasses a method of treating gastroesophageal reflux disease and of treating conditions caused by or contributed to by gastric hypersecretion. Conditions associated with hypersecretion in humans may include, but are not limited to, Zollinger-Ellison syndrome.

The present invention further encompasses a method of treating psoriasis while substantially reducing the adverse effects of racemic lansoprazole.

Utilizing the optically pure or substantially optically pure isomer of (−) lansoprazole results in enhanced efficacy, diminished adverse effects, and accordingly, an improved therapeutic index. It also provides more rapid onset and longer duration of the therapeutic effect. Moreover, the S(−) enantiomer exhibits fewer drug-drug interactions and shows less variation in the patient population between so-called good metabolizers and poor metabolizers. It is therefore, more desirable to use the (−) isomer of lansoprazole than to administer the racemic mixture because predictability of an effective and safe dose for an individual patient is greater. The S(−) enantiomer of lansoprazole is metabolized by both CYP2D6 and CYP3A4; the R(+) enantiomer is metabolized only by CYP2D6, which is the polymorphically expressed enzyme. Because it is metabolized by both enzymes, the S(−) shows less variability in the patient population and a more predictable dosage regimen. Surprisingly, the S-(−) isomer also shows a longer duration, a higher AUC and a more rapid onset as a result of lower first pass metabolism.

The term “adverse effects” includes, but is not limited to, hepatocellular neoplasia, gastrin hypersecretion, gastric carcinoids, headache, diarrhea, skin alterations and drug-drug interactions.

The term “substantially free of its (+) stereoisomer” as used herein means that the compositions contain at least 90% by weight of (−) lansoprazole and 10% by weight or less of (+) lansoprazole. In a more preferred embodiment the term “substantially free of the (+) isomer” means that the composition contains at least 99% by weight of (−) lansoprazole, and 1% or less of (+) lansoprazole. These percentages are based upon the total amount of lansoprazole in the composition. The terms “substantially optically pure (−) isomer of lansoprazole” or “substantially optically pure (−) lansoprazole” and “optically pure (−) isomer of lansoprazole” and “optically pure (−) lansoprazole” are also encompassed by the above-described amounts.

The term “treating ulcers” as used herein means treating, alleviating or palliating such conditions, and thus providing relief from the symptoms of nausea, heartburn, post-prandial pain, vomiting, and diarrhea.

The term “a method for treating gastroesophageal reflux diseases in a human” as used herein means treating, alleviating or palliating the conditions that result from the backward flow of the stomach contents into the esophagus.

The term “treating a condition caused, or contributed to, by gastric hypersecretion in a human” as used herein means treating, alleviating or palliating such disorders associated with hypersecretion, thus providing relief from the symptoms of the aforementioned conditions. Zollinger-Ellison Syndrome is among the conditions caused by or contributed to by hypersecretion.

The term “treating psoriasis” as used herein means treating, alleviating or palliating the condition, and thus providing relief from the symptoms of pruritis, epidermal scaling, itching and burning.

The magnitude of a prophylactic or therapeutic dose of (−) lansoprazole in the acute or chronic management of disease will vary with the severity of the condition to be treated and the route of administration. The dose and perhaps the dose frequency will also vary according to the age, body weight and response of the individual patient. In general, the total daily dose range for (−) lansoprazole for the conditions described herein is from about 10 mg to about 180 mg in single or divided doses. Preferably a daily dose range should be about 15 mg to about 60 mg in single or divided doses. In managing the patient, the therapy should be initiated at a lower dose, perhaps at about 10 mg to about 15 mg and increased up to about 60 mg or higher depending on the patient's global response. It is further recommended that children and patients over 65 years and those with impaired renal or hepatic function, initially receive low doses, and that they be titrated based on individual response(s) and blood level(s). It may be necessary to use dosages outside these ranges in some cases as will be apparent to those skilled in the art. Further, it is noted that the clinician or treating physician will know how and when to interrupt, adjust, or terminate therapy in conjunction with individual patient response. The terms “an amount sufficient to alleviate or palliate ulcers but insufficient to cause said adverse effects,” “an amount sufficient to alleviate the symptoms of gastroesophageal reflux but insufficient to cause said adverse effects,” “an amount sufficient to alleviate gastric hypersecretion but insufficient to cause said adverse effects” and “an amount sufficient to treat psoriasis” are encompassed by the above-described dosage amounts and dose frequency schedule.

The relative activity, potency and specificity of optically pure lansoprazole and racemic lansoprazole both as gastric antisecretory agents and plasma gastrin elevating agents can be determined by a pharmacological study in animals according to the method of Decktor et al. [J. Pharmacol. Exp. Ther. 249, 1-5 (0.1989)]. The test provides an estimate of relative activity, potency and, through a measure of specificity, an estimate of therapeutic index. Fasted rats, implanted with a gastric cannula, receive single oral or parenteral doses of (+) lansoprazole, (−) lansoprazole or racemate, 1 hour before collection of gastric juice over a four hour period. Acid output and pH are then determined on each sample. Dose response evaluations are performed with each compound to determine the lowest dose which inhibits acid output by at least 95% and maintains gastric pH above 7.0. Plasma gastrin levels are then determined in a second group of rats treated with the doses selected in the first series of tests. Blood samples are taken for analyses over the five hour period after dosing, and both peak level as well as area-under-the-curve analyses of the gastrin responses are made. These responses are then analyzed statistically using Student's “t” test to assess whether equivalent antisecretory doses show differences in gastrin responses.

Any suitable route of administration may be employed for providing the patient with an effective dosage of (−) lansoprazole. Rectal, parenteral (subcutaneous, intramuscular, intravenous), transdermal, topical and like forms of administration are possible; oral administration is preferred. Oral dosage forms include tablets, troches, dispersions, suspensions, solutions, capsules, and the like.

The pharmaceutical compositions of the present invention comprise (−) lansoprazole as the active ingredient, or a pharmaceutically acceptable salt thereof, and may also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable non-toxic bases. Since the compound of the present invention is a weak acid and is unstable at low pH, salts may be prepared from pharmaceutically acceptable non-toxic bases including inorganic and organic bases. Suitable pharmaceutically acceptable base addition salts for the compound of the present invention include metallic salts of aluminum, calcium, lithium, magnesium, potassium, sodium, titanium and zinc or organic salts made from lysine, N,N′-dibenzylethylenediamine, chloroprocaine, choline, diethanolamine, ethylenediamine, meglumine (N-methylglucamine) and procaine. If any salt is to be used, sodium salts are preferred.

The compositions of the present invention include suspensions, solutions, elixirs or solid dosage forms. Carriers such as starches, sugars, and microcrystalline cellulose, diluents, granulating agents, lubricants, binders, disintegrating agents, and the like are suitable in the case of oral solid preparations (such as powders, capsules, and tablets), and oral solid preparations are preferred over the oral liquid preparations. It has been found that the inclusion of basic salts of calcium and magnesium in the compositions allows the preparation of tablets and capsules having lansoprazole in a non-salt form and yet retaining good stability. If desired, tablets and granules may be coated by standard aqueous or nonaqueous techniques. Oral dosage forms suitable for lansoprazole are described in U.S. Pat. No. 5,035,899 and in PCT applications WO96/01624, WO97/12580 and WO97/25030, the disclosures of which are incorporated herein by reference.

In addition to the common dosage forms set out above, the compounds of the present invention may also be administered by controlled release formulations, which are well known in the art. Compositions suitable for rectal administration are described in European Application 645140, the disclosure of which is incorporated herein by reference.

Pharmaceutical compositions of the present invention suitable for oral administration may be presented as discrete units such as capsules, cachets, or tablets, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made by molding in a suitable machine, a mixture of the powdered compound moistened with an inert liquid diluent. Desirably, each tablet contains from about 5 mg to about 180 mg of the active ingredient, and each cachet or capsule contains from about 5 mg to about 180 mg of the active ingredient. Most preferably, the tablet, cachet or capsule contains one of three dosages: about 15 mg, about 30 mg or about 60 mg of (−) lansoprazole for oral administration.

The invention is further defined by reference to the following examples describing in detail the preparation of the compositions of the present invention, as well as their utility. It will be apparent to those skilled in the art that many modifications, both to materials and methods, may be practiced without departing from the purpose and interest of this invention.

### EXAMPLES

### Example 1

### Tablets

Composition per tablet:

| | | |
|---|---|---|
| S (−) lansoprazole | 30 | mg |
| Precipitated calcium carbonate | 50 | mg |
| Corn Starch | 40 | mg |
| Lactose | 73.4 | mg |
| Hydroxypropylcellulose | 6 | mg |
| Magnesium stearate | (0.05 | ml) |
| Total | 200.0 | mg |

### Example 1

S(−) Lansoprazole, precipitated calcium carbonate, corn starch, lactose and hydroxypropylcellulose are mixed together, water is added, and the mixture is kneaded, then dried in vacuum at 40° C. for 16 hours, ground in a mortar and passed through a 16-mesh sieve to give granules. To this is added magnesium stearate and the resultant mixture is made up into tablets each weighing 200 mg on a rotary tableting machine.

### Example 2

### Granules

Composition per tablet:

| | | |
|---|---|---|
| S (−) lansoprazole | 30 | mg |
| Magnesium carbonate | 20 | mg |
| Corn Starch | 80 | mg |
| Microcrystalline cellulose | 20 | mg |
| Carboxymethylcellulose calcium | 10 | mg |
| Hydroxypropylcellulose | 10 | mg |
| Pluronic F68 | 4 | mg |
| Lactose | 26 | mg |
| Water | (0.05 | ml) |
| Total | 200 | mg |

### Example 2

The ingredients above are mixed well in the proportions shown, water is added, and the mixture is kneaded and granulated in an extruder granulator (screen size 1.0 mm φ). The granules are immediately converted to spherical form in a spheronizer. The spherical granules are then dried under vacuum at 40° C. for 16 hours and passed through round sieves to give 12- to 42-mesh granules.

### Example 3

### Capsules

Enteric coating composition:

| | | |
|---|---|---|
| Eudragit L-30D | 138 | mg (solids 41.4 mg) |
| Talc | 4.1 | mg |
| Polyethylene glycol | 12.4 | mg |
| 5000 | | |
| Tween 80 | 2.1 | mg |
| Water | 276 | μl |

Composition of enteric granules:

| | | |
|---|---|---|
| Granules of Example 5 | 200 | mg |
| Enteric coat | 60 | mg |
| Total | 260 | mg |

Composition per capsule:

| | | |
|---|---|---|
| Enteric granules | 260 | mg |
| No. 1 hard capsule | 76 | mg |
| Total | 336 | mg |

### Example 3

Enteric granules are produced by coating the granules obtained in Example 2 with the enteric coating composition shown using a fluidized bed granulator under conditions such that the inlet air temperature is 50° C. and the granule temperature is about 40° C. Number 1 hard capsules are filled with the enteric granules thus obtained in an amount of 260 mg per capsule using a capsule filling machine.

Tablets of other strengths may be prepared by altering the ratio of active ingredient to the excipients or to the final weight of the tablet. An enteric coating, such as the polyacrylate Eudragit L® and Eudragit S® series, is applied by spray coating the tablets, preferably with an aqueous dispersion of the coating polymer.

1-12. (canceled)

13. A method of treating ulcers with lansoprazole which comprises administering to a human a therapeutically effective amount of optically pure S(−)isomer of lansoprazole, or a pharmaceutically acceptable salt thereof.

14. The method of claim 13, wherein the amount of S(−) lansoprazole or a pharmaceutically acceptable salt thereof is greater than approximately 90% by weight of the total weight of lansoprazole.

15. The method of claim 13, wherein S(−) lansoprazole is administered orally in the form of a tablet or capsule.

16. The method of claim 15, wherein the amount of S(−) lansoprazole or a pharmaceutically acceptable salt thereof administered is from about 5 mg to about 180 mg per day.

17. A method of treating gastroesophageal reflux disease which comprises administering to a human a therapeutically effective amount of optically pure S(−)isomer of lansoprazole, or a pharmaceutically acceptable salt thereof.

18. The method of claim 17, wherein the amount of S(−) lansoprazole or a pharmaceutically acceptable salt thereof is greater than approximately 90% by weight of the total weight of lansoprazole.

19. The method of claim 17, wherein S(−) lansoprazole is administered orally in the form of a tablet or capsule.

20. The method of claim 19, wherein the amount of S(−) lansoprazole or a pharmaceutically acceptable salt thereof administered is from about 5 mg to about 180 mg per day.

21. A method of treating a condition caused by or contributed to by gastric hypersecretion which comprises administering to a human a therapeutically effective amount of optically pure S(−)isomer of lansoprazole, or a pharmaceutically acceptable salt thereof.

22. The method of claim 21, wherein the amount of S(−) lansoprazole or a pharmaceutically acceptable salt thereof is greater than approximately 90% by weight of the total weight of lansoprazole.

23. The method of claim 21, wherein S(−) lansoprazole is administered orally in the form of a tablet or capsule.

24. The method of claim 23, wherein the amount of S(−) lansoprazole or a pharmaceutically acceptable salt thereof administered is from about 5 mg to about 180 mg per day.

25. The method according to claim 21, wherein said condition is Zollinger-Ellison Syndrome.

26. A method of treating psoriasis which comprises administering to a human a therapeutically effective amount of optically pure S(−)isomer of lansoprazole, or a pharmaceutically acceptable salt thereof.

27. The method of claim 26, wherein the amount of S(−) lansoprazole or a pharmaceutically acceptable salt thereof is greater than approximately 90% by weight of the total weight of lansoprazole.

28. The method of claim 26, wherein S(−) lansoprazole is administered orally in the form of a tablet or capsule.

29. The method of claim 28, wherein the amount of S(−) lansoprazole or a pharmaceutically acceptable salt thereof administered is from about 5 mg to about 180 mg per day.
